(19) United States
(12) Patent Application Publication
(10) Pub. No.: US 2026/0276766 A1
(43) Pub. Date: Sep. 17, 2026

(54) METHOD AND APPARATUS FOR PERFORMING ACCELERATED MAGNETIC RESONANCE IMAGING WITH REDUCED OVERSAMPLING AT LOW SPATIAL FREQUENCIES

(71) Applicant: COMMISSARIAT À L'ENERGIE ATOMIQUE ET AUX ENERGIES ALTERNATIVES, PARIS (FR)

(72) Inventors: Philippe CIUCIU, GIF-SUR-YVETTE (FR); Chaithya GILIYAR RADHAKRISHNA, GIF-SUR-YVETTE (FR)

(21) Appl. No.: 19/168,323

(22) PCT Filed: May 22, 2024
(86) PCT No.: PCT/EP2024/064108

§ 371 Date: Sep. 23, 2025

(30) Foreign Application Priority Data
May 22, 2023 (EP) 23305808.0

(51) Int. Cl. G01R 33/48; A61B 5/055; G01R 33/54; G01R 33/561

(52) U.S. Cl. CPC G01R 33/482; A61B 5/055; G01R 33/543; G01R 33/561

(57) ABSTRACT

A method of performing magnetic resonance imaging of a body using a magnetic resonance imaging scanner the method includes applying to the body a time-varying magnetic field gradients defining a continuous trajectory in k-space complying with a set of constraints including constraints on maximum amplitude and maximum slew rate of the time-varying magnetic field gradients, such that sampling points (KS) belonging to the trajectory define a pseudo-random sampling of the k-space, approximating a predetermined target sampling density, the trajectory in k-space minimizing, subject to the set of constraints, a cost function defined by the difference between a first term, called attraction term, promoting adherence of the distribution of sampling points in k-space to the predetermined target sampling density, and a second term, called repulsion term, promoting separation in k-space between sampling points, the repulsion term being expressed as a sum of contributions corresponding to respective pairs of sampling points; wherein the trajectory in k-space defines a Cartesian sampling of a central region(S) of the k-space.

The invention relates to a method and apparatus for performing accelerated magnetic resonance imaging while minimizing the adverse impact of oversampling in the central region of the k-space, i.e. at low spatial frequencies.

Magnetic Resonance Imaging (MRI) is one of the most powerful imaging techniques used in clinical routine today, but remains a lengthy procedure, particularly when the acquisition of large and/or high resolution images, comprising several millions of pixels, is sought. For instance, acquiring a three-dimensional image of a human brain with a field of view of 205×205×52 mm³ and a 200 μm resolution using a T2* sequence at an acceptable signal-to-noise ratio (SNR) of 7.6 may require an acquisition time of about three hours for a short repetition time (TR≅20 ms), which is clearly unacceptable for clinical purposes.

This is because MRI images are obtained by sampling the so-called “k-space” domain, the spatial frequency spectrum of the image, which is related to the physical space by a multidimensional Fourier transform. Sampling theory teaches that the sampling frequency should be at least twice the highest frequency contained in the signal to be sampled (which, in the case of MRI, is the multidimensional Fourier transform of the body to be imaged), otherwise aliasing artifacts will appear; this is known as the Nyquist, or Nyquist-Shannon, criterion. As a consequence, using conventional acquisition schemes, the number of k-space samples must be at least equal to the number of pixels of the image to be reconstructed. Moreover, SNR requirements impose a minimum acquisition time for each sample.

Several techniques have been developed in order to reduce the acquisition time while avoiding artifact (“accelerated MRI”).

Some of these techniques, such as Simultaneous Multislice Imaging (SMS) and parallel MRI involve the use of specialized hardware comprising multiple receiver coils for acquiring magnetic resonance signals. Their implementation is therefore expensive. Moreover, they provide a limited acceleration, because image quality drops fast with the acceleration factor. Even using both techniques simultaneously, the combined acceleration factor does not exceed 8 in practice.

Other techniques are compatible with the use of a single receiving coil (even though the use of multiple coils is also possible), like Partial Fourier imaging, which exploits redundancies in k-space information, non-Cartesian k-space filling such as radial or spiral and Compressed Sensing (CS). While Partial Fourier techniques only offer very limited acceleration factors (typically lower than two), Compressed Sensing allows higher accelerations especially while imaging with high matrix size (either high resolution and small field of view or low resolution and large field of view), see [Haldar, 2014].

A review of CS MRI can be found in [Lustig et al, 2008].

Compressed Sensing techniques rely on three principles:
The image to be reconstructed must admit a sparse (or compressible) representation. Said differently, it must be possible to decompose it on a predetermined basis (e.g. a wavelet basis) such that only a small fraction of the decomposition coefficients is non-zero for strict sparsity or significantly greater than zero for compressibility. Typically, in the case of a noisy signal, a coefficient is considered significantly greater than zero if its absolute value is at least equal to the noise standard deviation. Alternatively, only a predetermined fraction of the coefficients-those having the greatest absolute value—may be kept. For instance, only the top 1% of the coefficient may be kept, resulting in a compression factor of 100.
Reconstruction must be performed using a nonlinear method promoting sparsity of the image representation, as well as consistency with the acquired samples.
The k-space must be under-sampled, in order to accelerate the acquisition, following a pseudo-random pattern. The under-sampling reduces the number of signal acquisitions, and therefore provides the required acceleration, while pseudo-randomness ensures that, in the sparsifying representation, subsampling artifacts are incoherent, i.e. decorrelated or noise-like. This incoherence property is extremely important and measures the degree of correlation between any pair of elements taken in the sparsifying (e.g., wavelets) and sensing (e.g., Fourier in CS-MRI) bases.

Compressed sensing often uses non-Cartesian pseudo-random sampling of k-space since this strategy provides improved incoherence (lower correlation between samples). Preferably, the pseudo-random sampling is non-uniform. This is because an image decomposition in a sparsifying basis like a wavelet transform consists of approximation and detail coefficients. The approximation coefficients encode low frequency information and thus are not sparse while the detail ones encode high frequency content and are sparse. Sparsity can be imposed to recover the detail coefficients while information (low frequency content) associated with the approximation ones must be collected in the data. Due to this we must adopt a variable density sampling approach that picks up more measurements in the low frequencies as compared to the high frequencies. Another view on that is to say that variable density sampling (VDS) is a theoretical means to minimize the number of collected samples to guarantee exact image reconstruction with high probability. In clinical application, this usually means using a variable sampling density which is highest near the center of the k-space (low spatial frequencies) and decreases toward high spatial frequencies.

From a purely theoretical point of view, the pseudo-random sampling could be obtained by drawing sampling points following a predefined probability distribution, corresponding to the required sampling density. But in practice, the short lifespan of MR signals requires samples to be acquired through segmented acquisitions along smooth trajectories which are defined by time-varying magnetic field gradients applied to the body to be imaged after the excitation of its nuclear spins by a radio-frequency (RF) pulse.

Let \vec{G} represent the magnetic field gradients applied to the body to be imaged. These magnetic field gradients defines a trajectory in the k-space which is expressed by:

$$\vec{k ( t )} = γ \cdot {\int }_{0}^{t} \vec{G ( τ )} d τ \qquad ( 1 )$$

Sampling is performed by acquiring the MRI signal generated by excited nuclear spins at predetermined times, which correspond to points along said trajectory.

Neither the gradient field amplitude ∥\vec{G(t)}∥ nor its slew rate can exceed respective limits Gmax and Smax, due to both hardware and, for clinical applications, physiological constraints. Therefore, only sufficiently regular trajectories are allowed.

These trajectories may be bi-dimensional in 2D MRI, when only nuclear spins within a thin slice of the body are excited, on three-dimensional in 3D MRI techniques, where the excitation concerns the whole body or a thick slab thereof. In the following, for the sake of simplicity, only the case of 2D trajectories will be considered; the invention, however, also applies to 3D and even 4D (i.e. dynamic) MRI.

An important feature of MRI is that the signal decays exponentially after the application of the exciting RF pulse, and typically vanishes. This limits the duration of signal acquisition, and therefore the length of each individual k-space trajectory. As a consequence, several excitation RF pulses, each followed by MRI signal acquisition along a respective elementary trajectory (or “shot”), are required to perform a full k-space sampling. The repetition time TR of these excitation RF pulses—which imposes an upper limit on the duration of the signal acquisition—also depends on the used imaging technique, and in particular on the type of contrast which is sought (T1, T2, T2* . . . ).

Commonly used k-space trajectories are parallel lines (leading to Cartesian sampling), spokes (straight lines radially diverging from the center of the k-space), rosettes, uniform- and variable-density spirals. All of them have been applied to Compressed Sensing, for instance by performing only a limited number of signal acquisitions over a Cartesian grid or by randomly sampling spokes, spirals or rosettes.

Better results, however, are achieved by using “non-parametric” trajectories that provide larger incoherence.

In particular, the so-called SPARKLING (“Spreading Projection Algorithm for Rapid K-space sampLING”) technique ([Boyer et al, 2016], [Lazarus et al. 2017], [Lazarus et al. 2019], [Chaithya et al. 2020], WO2019/048565) is based on the projection of a predetermined, usually non-uniform, Target Sampling Distribution (TSD) onto the set of “admissible” 2D or 3D curves, i.e. to all the curves representing trajectories obtainable without exceeding the limits on the values of the magnetic field gradients and the corresponding slew rate.

[Chaithya et al. 2022] discloses an improvement of SPARKLING, called MORE SPARKLING.

A drawback of some non-Cartesian MRI sampling schemes, such as SPARKLING, is that they vastly oversample the central part of the k-space, at a rate much higher than the Nyquist rate—sometimes up to 20 k-space samples per Nyquist voxel on average. Indeed, while high-spatial frequency components (“detail”, in a wavelet decomposition) can be undersampled and then recovered by sparsity constraints—and this is indeed at the heart of CS-MRI—low-spatial frequencies (“approximation” in a wavelet decomposition) must be fully sampled to ensure perfect recovery. However, in most non-Cartesian CS-MRI implementation, including SPARKLING, every shot is constrained to pass through the center of the k-space at the Echo time TE (Echo Time constraint, see [Chauffert et al. 2016]), notably in order to have a good target contrast and to allow the correction of motion artifacts, leading to oversampling, which is disadvantageous for at least two reasons. On the one hand, these extra k-space samples could be used to sample higher frequencies instead. On the other hand, as the k-space data are acquired as integration over different k-space trajectories, such repeated sampling of the same Nyquist voxel by trajectories of different paths can lead to accumulation of B₀ artifacts.

A partial solution of this problem is to update the TSD by taking the TE constraint into account and reducing the density around the center of the k-space to limit the number of samples per Cartesian k-space voxel. This, however, does not fully mitigate the problem, as several samples (one per shot) are still present at the center of k-space causing nearby regions to be sampled densely.

Even if the Echo Time constraint is removed (which is possible for sufficiently low scan times, albeit at the cost of a reduction of target contrast), oversampling can still occur if the shots are stuck in local minima. Furthermore, removing the Echo Time constraint can lead to sampling the center of k-space at different times, thereby destroying contrast and increasing B₀ artifacts.

The invention aims at overcoming, in full or in part, the above-mentioned drawback of the prior art. More particularly, it aims at providing a method of accelerated MRI ensuring that the central part of the k-space is sampled at or near the Nyquist criterion, avoiding both oversampling and undersampling.

According to the invention, this aim is achieved by modifying SPARKLING trajectories by introducing suitable affine constraints to ensure a substantially Cartesian sampling of a central region of the k-space. This approach will be called GoLF-SPARKLING as it stands for Gridding of Low Frequency (GoLF) SPARKLING.

An object of the invention is then a method of performing magnetic resonance imaging of a body using a magnetic resonance imaging scanner comprising a scanner bore, a primary coil, radio frequency coils, gradient coils) and a signal-processing unit, the method comprising the steps of:
a. positioning the body in a scanner bore where a static and substantially uniform magnetic field, called longitudinal field, oriented along a direction, called longitudinal direction, is established by the primary coil;
b. using all or part of the radio-frequency coils to transmit to said body at least one radio-frequency pulse adapted to excite nuclear spins inside said body;
c. after said or each said radio-frequency pulse, using the gradient coils to apply to said body time-varying magnetic field gradients defining a trajectory in k-space and simultaneously using all or part of the radio-frequency coils to acquire samples of a magnetic resonance signal emitted by the excited nuclear spin, each sample corresponding to a point (KS) of the k-space belonging to said trajectory; and
d. using the signal processing unit) to apply a nonlinear reconstruction algorithm to the acquired samples to reconstruct a magnetic resonance image of said body;
wherein said trajectory in k-space is a continuous trajectory complying with a set of constraints including constraints on maximum amplitude and maximum slew rate of said time-varying magnetic field gradients, such that the points of the k-space corresponding to the samples, called sampling points, define a pseudo-random sampling of the k-space, approximating a predetermined target sampling density, said trajectory in k-space minimizing, subject to said set of constraints, a cost function defined by the difference between a first term, called attraction term, promoting adherence of the distribution of sampling points in k-space to said predetermined target sampling density, and a second term, called repulsion term, promoting separation in k-space between sampling points, said repulsion term being expressed as a sum of contributions corresponding to respective pairs of sampling points;
characterized in that said trajectory in k-space defines a Cartesian sampling of a central region of the k-space.

Another object of the invention is a computer-implemented method of computing a trajectory in k-space to be used for sample acquisition in magnetic resonance imaging, the method comprising the steps of:
i. determining, or receiving from a user input, a number N_{c}≤1 of shots constituting the trajectory and a number Nₛ>1 of sampling points along each said shot;
ii. determining, or receiving from a user input, a predetermined target sampling density;
iii. determining, or receiving from a user input, a set of constraints including constraints on amplitudes of discrete time derivatives of the trajectory;
iv. computing said trajectory by minimizing, subject to said set of constraints, a cost function defined by the difference between a first term, called attraction term, promoting adherence of the distribution of sampling points in k-space to said predetermined target sampling density, and a second term, called repulsion term, promoting separation in k-space between sampling points, said repulsion term being expressed as a sum of contributions corresponding to respective pairs of sampling points;
characterized in that said trajectory in k-space defines a Cartesian sampling of a central region of the k-space.

Another object of the invention is a computer program comprising instructions which, when the program is executed by a computer, cause the computer to carry out said method of computing a trajectory in k-space to be used for sample acquisition in magnetic resonance imaging.

Yet another object of the invention is a set of driving signals for gradient coils of a magnetic resonance imaging scanner which, when applied to said gradient coils, drive them to generate time-varying magnetic field gradients defining a trajectory in k-space passing through a plurality of points called sampling points defining a pseudo-random sampling of the k-space, approximating a predetermined target sampling density; wherein said trajectory:
is continuous and complies with a set of constraints including constraints on maximum amplitude and maximum slew rate of said time-varying magnetic field gradients;
minimizes, subject to said set of constraints, a cost function defined by the difference between a first term, called attraction term, promoting adherence of the distribution of sampling points in k-space to said predetermined target sampling density, and a second term, called repulsion term, promoting separation in k-space between sampling points, said repulsion term being expressed as a sum of contributions corresponding to respective pairs of sampling points;
characterized in that said trajectory in k-space defines a Cartesian sampling of a central region of the k-space A further object of the invention is a magnetic resonance imaging scanner comprising:
a scanner bore inside which a body can be positioned;
a primary coil configured to establish in the scanner bore a static and substantially uniform magnetic field, called longitudinal field, oriented along a direction, called longitudinal direction;
radio frequency coils configured to transmit to said body at least one radio-frequency pulse adapted for exciting nuclear spins inside said body and to acquire samples of a magnetic resonance signal emitted by the excited nuclear spin;
gradient coils configured to apply to said body time-varying magnetic field gradients defining a trajectory in k-space;
a signal processing unit to apply a nonlinear reconstruction algorithm to the acquired samples of the magnetic resonance signal for reconstructing a magnetic resonance image of said body; and
a controller configured to generate driving signals for the radio frequency coils and for the gradient coils;
wherein the controller is configured to generate said driving signals for the gradient coils to generate said time-varying magnetic field gradients such that said a trajectory in k-space passes through a plurality of points called sampling points defining a pseudo-random sampling of the k-space, approximating a predetermined target sampling density, said trajectory being continuous and compliant with a set of constraints including constraints on maximum amplitude and maximum slew rate of said time-varying magnetic field gradients, said trajectory in k-space also minimizing, subject to said set of constraints, a cost function defined by the difference between a first term, called attraction term, promoting adherence of the distribution of sampling points in k-space to said predetermined target sampling density, and a second term, called repulsion term, promoting separation in k-space between sampling points, said repulsion term being expressed as a sum of contributions corresponding to respective pairs of sampling points;
and wherein the controller is also configured to generate driving signals for the radio frequency coils such that they acquire said samples of a magnetic resonance signal emitted by the excited nuclear spin in correspondence to the sampling points of the trajectory in k-space;
characterized in that said trajectory in k-space defines a Cartesian sampling of a central region of the k-space.

A further object of the invention is a non-transitory computer-readable information storage support—e.g. a magnetic storage support such as a hard disk or a tape; an optical storage support such as a CD or DVD or a semiconductor non-volatile memory such as a flash memory or solid-state hard disk (SSHD), or the like—storing said computer program and or said set of driving signals.

Additional features and advantages of the present invention will become apparent from the subsequent description, taken in conjunction with the accompanying drawings, which show:

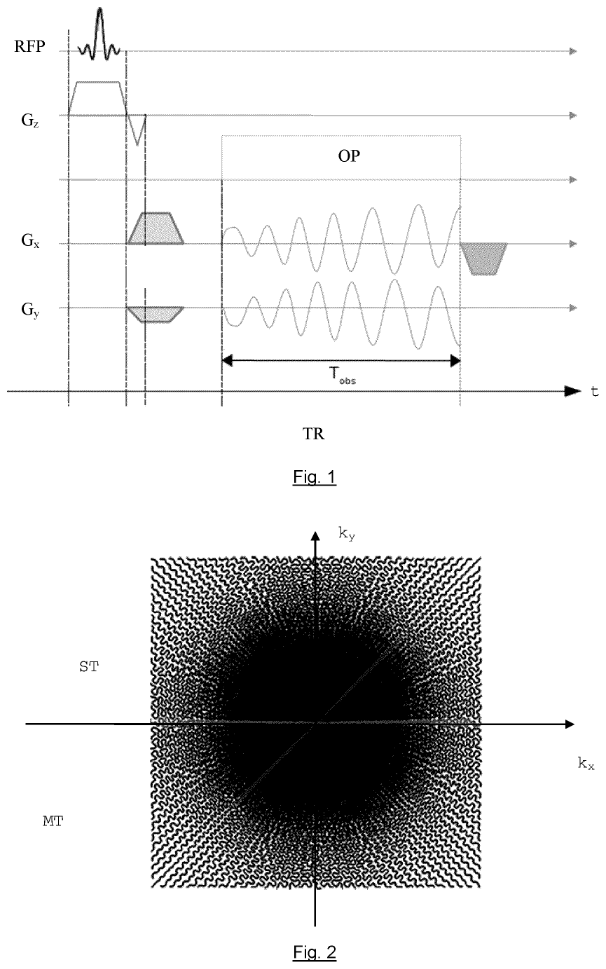

FIG. 1, the chronogram of a modified GRE (Gradient Recalled Echo) pulse sequence to play implementing a SPARKLING trajectory according to the prior art or a GoLF SPARKLING trajectory according to an embodiment of the invention.

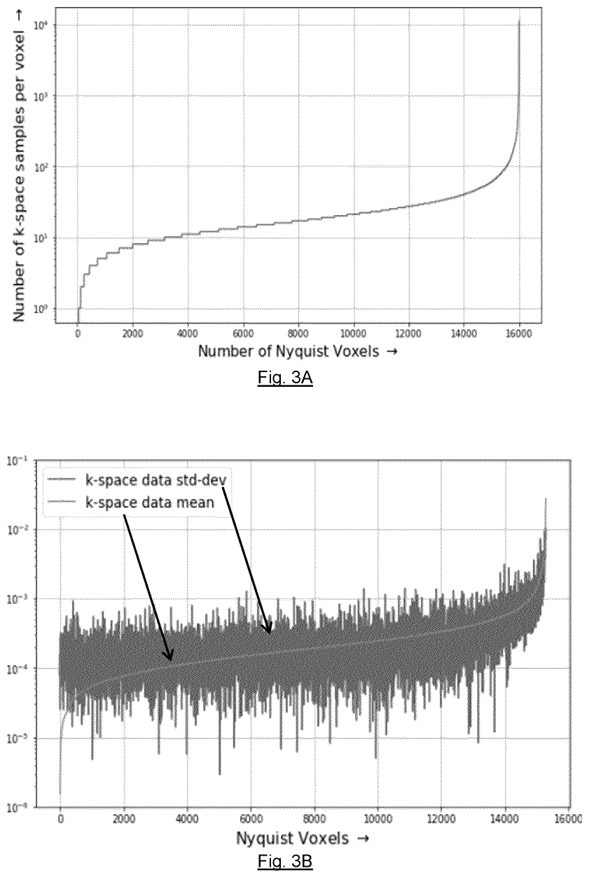

FIG. 2, an exemplary 2D SPARKLING trajectory according to the prior art;

FIGS. 3A and 3B, plots illustrating the oversampling of low spatial frequencies obtained using a SPARKLING trajectory according to the prior art;

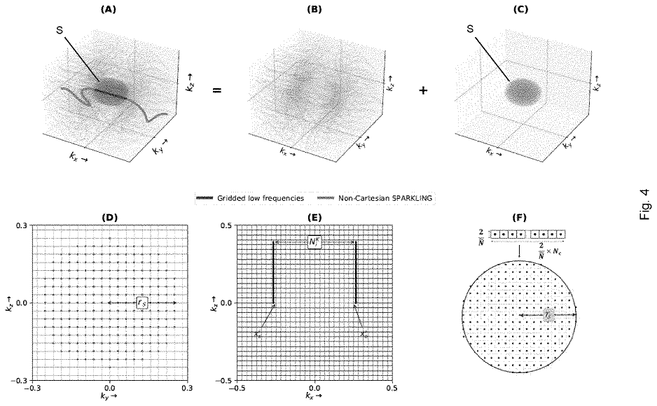

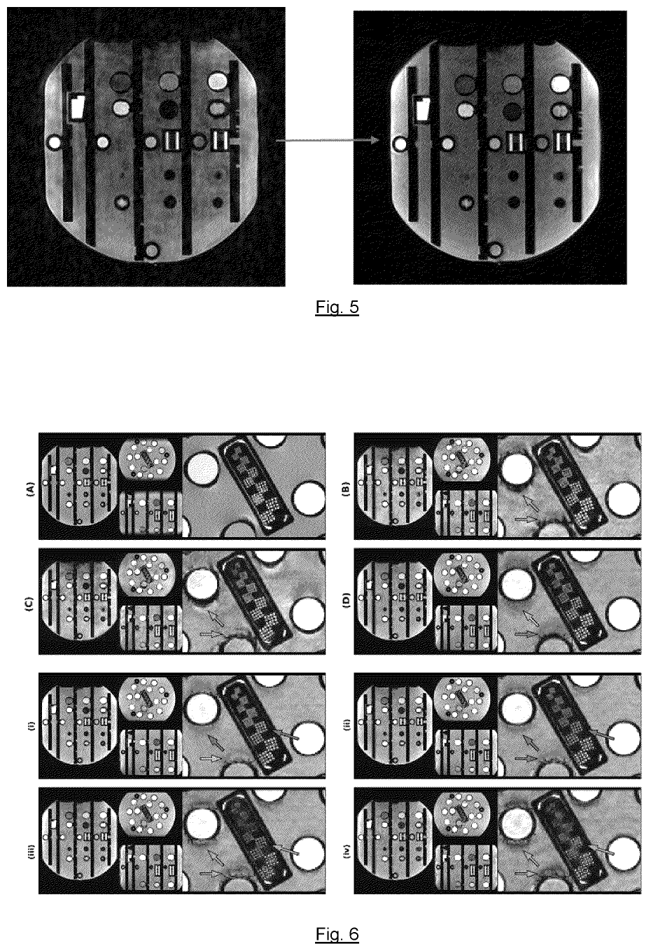

FIG. 4, an example of GoLF-SPARKLING trajectories according to respective embodiments of the invention;

FIGS. 5 and 6, experimental results obtained on a phantom illustrating the technical effect of the invention;

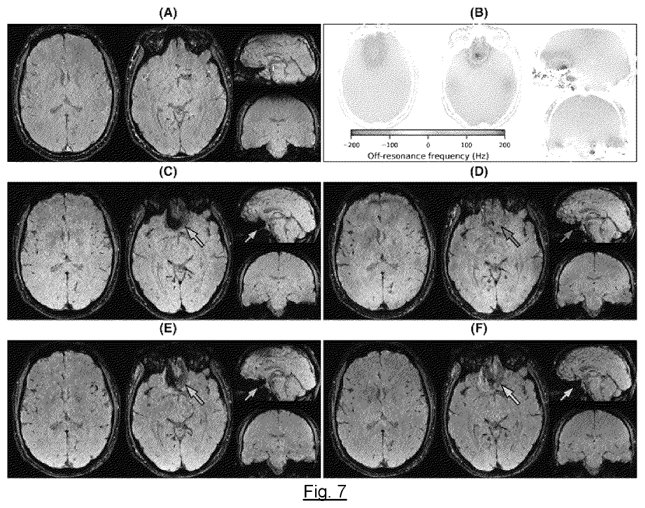

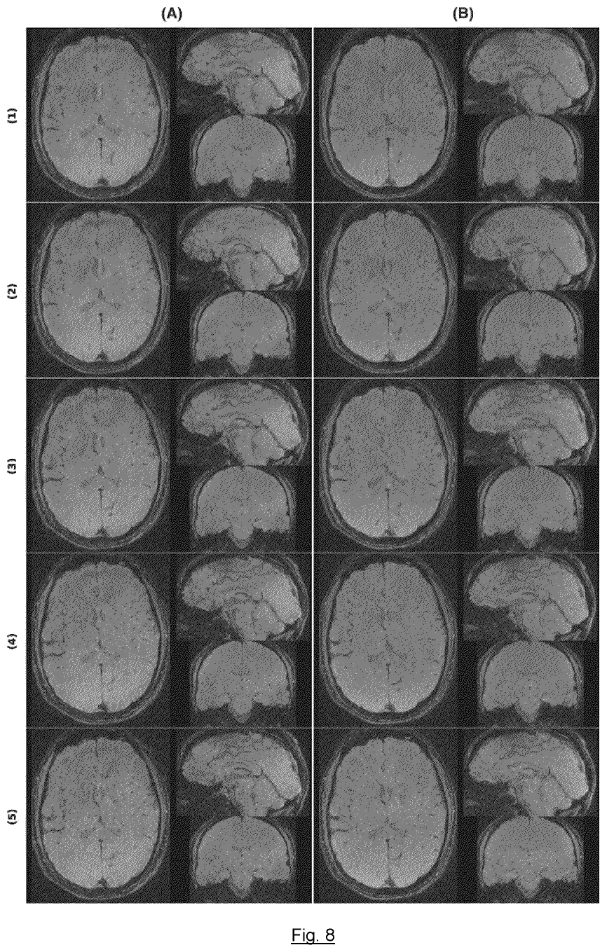

FIGS. 7 and 8, experimental results obtained on healthy volunteer, also illustrating the technical effect of the invention; and

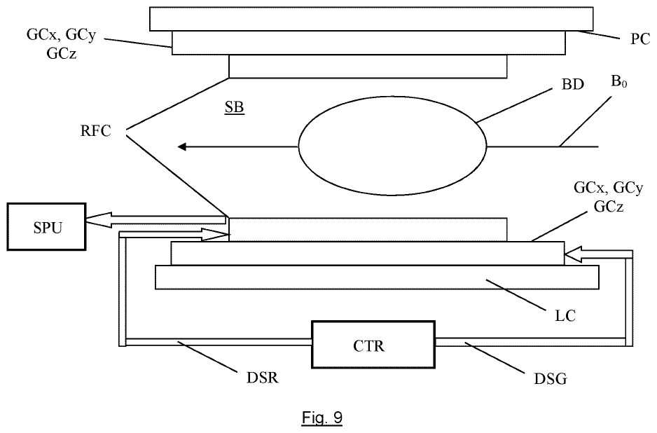

FIG. 9, a block diagram of a MRI scanner according to an embodiment of the invention.

FIG. 1 shows the chronogram of a pulse sequence—more particularly a Gradient Recalled Echo (GRE) sequence for T2* weighted anatomical imaging—suitable for implementing either a SPARKLING k-space trajectory according to the prior art or a GoLF-SPARKLING one.

It is supposed that a body to be imaged (e.g. the head of a patient) is placed in a static and homogeneous magnetic field B₀ oriented along a “longitudinal” direction z. This magnetic field induces a partial alignment of the spins of the atomic nuclei of the body. The aligned nuclear spins may be excited (i.e. flipped) by a radio-frequency pulse RFP at a suitable frequency (Larmor frequency), depending on the values of the magnetic field B₀ and of the magnetic momentum of the nuclei. In 2D sequences, as the one illustrated on the figure, a magnetic field gradient G_{z}—i.e. a magnetic field oriented along the z direction, whose magnitude varies linearly along this same direction—is also applied. This has the effect of making the Larmor frequency variable along z; as a consequence, only the nuclear spins within a thin slice of the body are resonant with the RF pulse and can be excited. As known in the art of MRI, this “slice selection” gradient pulse is followed by a short negative blip of the G_{z} magnetic field gradient (“refocusing gradient”) which compensates for a dispersion of the nuclear spin orientations in the my plane, perpendicular to the z direction.

Due to the use of a slice selection gradient G_{z}, only a 2D image of the selected slice of the body is acquired, which requires sampling of a 2D kaki plane of the k-space; in the following, the expression “k-space” will be used to designate both the three-dimensional kₓk_{y}k_{z} space and a two-dimensional plane within it. In alternative embodiments, no slice selection gradient is used, and a domain of the 3D kₓk_{y}k_{z} space will have to be sampled.

A trajectory in the k-space (more precisely, in the 2D kₓk_{y} plane) is defined by playing Gₓ and G_{y} gradients after the end of the RF excitation pulse. It is important to underline that the applied magnetic field is always oriented along the z direction, but its magnitude shows linear variations along the x and y directions. First of all, Gₓ and G_{y} pulses are applied to reach a suitable point on the boundary of the region of the kₓk_{y} plane to be sampled. Then “arbitrary” Gₓ and G_{y} waveforms are played, defining a winding trajectory with an overall radial orientation, directed toward the center of the kₓk_{y} plane.

In “3D” implementations without slice selection gradient, a G_{z} waveform similar to Gₓ and G_{y} will be applied to reach a suitable point of the boundary of the 3D domain in the kₓk_{y}k_{z} space and to define a three-dimensional trajectory in k-space.

While the gradient waveforms are played, samples of the MRI signal emitted by the excited nuclei are acquired by one or more radio-frequency coils connected to a suitable acquisition circuit including an analog-to-digital (ADC) converter. The acquisition period, whose duration T_{obs} is limited by the decay of the MRI signal, is designated by reference OP on FIG. 1. After the end of the acquisition period, a final Gₓ gradient pulse (“spoiling gradient”) is applied to cancel residual transversal magnetization. The repetition time TR corresponds to the interval beginning just before the radio-frequency pulse RFP and ending at the end of the spoiling gradient pulse.

This sequence is repeated several times with different gradient waveforms defining respective k-space trajectories which, together, provide the required k-space sampling. The ensemble constituted by an excitation RF pulse and the associated gradient waveforms is called a “shot”; each shot corresponds to an elementary trajectory. FIG. 2 shows a 2D “multishot” SPARKLING trajectory MT comprising N_{c}=60 radially-oriented elementary trajectories or shots ST, inhomogeneously sampling the kₓk_{y} plane.

In practice, the magnetic field gradients Gₓ, G_{y}, G_{z} undergo stepwise changes at discrete time-points separated by intervals of duration Δt (“gradient raster time”). Sampling is also performed at regular intervals of duration dt (“ADC dwell time”). The ADC dwell time δt is preferably lower than, or at most equal to, the gradient raster time (δt≤Δt) so as to allow collecting several samples between two consecutive gradient steps. At the same time, reducing the ADC dwell time beyond a certain limit decreases the SNR to an unacceptable level. Therefore, for each specific embodiment of the invention there is an optimal value for δt which can be found.

Let kᵢ(1) the position, in the k-space, of the starting point of the trajectory associated to the i^{th} shot. A first sample of the MRI signal is acquired in correspondence to this point. The other sampling points correspond to k-space positions given by:

$${k}_{i} ( m ) = {k}_{i} ( 1 ) + γ ( {\sum }_{j = 1}^{q} {G}_{i} ( j ) * Δ t + {G}_{i} ( q + 1 ) * r ) \qquad ( 2 )$$

where m∈[2:M] is an integer index, M being the overall number of samples acquired along the trajectory, and q and are respectively the modulus and the rest of the Euclidean division of the acquisition time by:

$${t}_{ADC , m} = ( m - 1 ) * δ t = q * Δ t + r \qquad ( 3 )$$

If δt=Δt, then r=0 and the number of ADC samples matches the number of gradient time steps. If δt<Δt, then the number of ADC samples is larger than the number of gradient time steps.

“SPARKLING” relies on an optimization-based method that consists of projecting a target sampling distribution over a set of discrete pushforward measures, in particular supported by smooth trajectories [Lazarus et al, 2017; Boyer et al, 2016]. Mathematically, this problem can be cast as a non-convex variational optimization problem under possibly non-convex constraints [Boyer et al, 2016]. The constraints are typical expressed by maximal acceptable values for the gradient amplitude and slew rate, but additional affine constraints may also be used—e.g. imposing that the trajectory passes through a particular point of the k-space at a defined time point (e.g. for echo time definition).

According to ([Boyer et al, 2016], [Lazarus et al. 2017], [Lazarus et al. 2019] and [Chaithya et al. 2020], a SPARKLING trajectory is computed by minimizing, subject to a set of constraints, a cost function defined by the difference between two terms: an “attraction term”, which promotes adherence of the distribution of sampling points in k-space to a predetermined (usually non-uniform) target sampling density, and a “repulsion term”, promoting separation in k-space between sampling points. This can be written as:

$$\hat{K} = {arg min}_{K ∈ {Q}^{N c}} {F}_{p} ( K ) = {F}_{p}^{a} ( K ) - {F}_{p}^{r} ( K ) \qquad ( 4 )$$

$$Where$$

$${F}_{p}^{a} ( K ) = {arg min}_{K ∈ {Q}^{N c}} \frac{1}{p} {\sum }_{i = 1}^{p} {\int }_{Ω} ‖ x - K [ i ] ϱ ( x ) ‖ dx \qquad ( 5 )$$

is the attraction term,

$${F}_{p}^{r} ( K ) = \frac{1}{2 {p}^{2}} {\sum }_{1 \leq i , j \leq p} ‖ K [ i ] - K [ j ] ‖ \qquad ( 6 )$$

is the repulsion term and:
N=N_{c}·Nₛ is the number of sampling points along the trajectory, N_{c}≥1 being the number of shots, or elementary trajectories, constituting the whole trajectory and Nₛ>1 the number of sampling points of each shot;
Q^{N}^{_{c}} is a set of curves in k-space comprising N_{c} shots, or segments, each comprising Nₛ points and complying with the constraints;

$$K = {( {k}_{i} )}_{i = 1}^{{N}_{c}}$$

is the K-space sampling pattern composed of N_{C}=shots kᵢ(t);
Ω is a sampling region in k-space, normalized to [−1, 1]³ (for 3D trajectories);
x represents a generic point of the sampling region Ω;
ρ is said predetermined target sampling density;
∥⋅∥ is a norm, preferably the L₂ norm.

The set of constraints can be defined as follows

$${Q}_{A , b}^{{N}_{c}} = \{ \begin{matrix}∀ i = 1 … {N}_{c} , {k}_{i} ∈ {Ω}^{Ns} \\ {A}_{i} {k}_{i} = \underline{{b}_{i}} \\ {‖ {k}_{i} ‖}_{∞} \leq 1 , {‖ {\dot{k}}_{i} ‖}_{2 , ∞} \leq α , {‖ {\ddot{k}}_{i} ‖}_{2 , ∞} \leq β\end{matrix} \} \qquad ( 7 )$$

where:
\dot{k}ᵢ, \ddot{k}ᵢ are, respectively, the first and second discretized time derivative of the trajectory in k-space;
For an arbitrary three-dimensional vector C,

$${\vert c ‖}_{2 , ∞} = {sup}_{0 \leq n \leq Ns - 1} \sqrt{{\vert {c}_{x} [ n ] \vert}^{2} + {\vert {c}_{y} [ n ] \vert}^{2} + {\vert {c}_{z} [ n ] \vert}^{2}} ;$$

$${{A}_{i} ( {a}_{i , 1} \vert {a}_{i , 2} \vert … \vert {a}_{i , {c}_{i}} )}^{T} ∈ {\{ 0 , 1 \}}^{3 {c}_{i} \times {N}_{s}} with$$

$${a}_{i , j} = [ \begin{matrix}{a}_{i , j}^{x} [ 1 ] & {a}_{i , j}^{y} [ 1 ] & {a}_{i , j}^{z} [ 1 ] \\ ⋮ & ⋮ & ⋮ \\ {a}_{i , j}^{x} [ {N}_{s} ] & {a}_{i , j}^{y} [ {N}_{s} ] & {a}_{i , j}^{z} [ {N}_{s} ]\end{matrix} ]$$

and bᵢ=(b_{i,1}, . . . , b_{i,c}_{ᵢ})^{T}∈ [symbol] ^{3c}^{ᵢ}, where j∈{1, . . . cᵢ}, cᵢ being the number of affine constraints on the i^{th} k-space shot, model the constraints. More precisely, Aᵢ se
α, β are normalized parameters expressing hardware constraints on gradient amplitude and gradient slew rate; and
   Ω^{N}^{ₛ} represents Nₛ points in Ω

Usually, the trajectory design process starts with an initialization trajectory, formed e.g. by N_{c} radial spokes, then the optimization problem (4) is solved under constraints (7) by the projected gradient descent algorithm, as discussed in [Chaithya et al. 2020].

Each individual shot of the trajectory of FIG. 2 passes through the center of the k-space, resulting in a sampling of the central (low-frequency) region of this space at a rate much higher than the Nyquist rate. For instance, as illustrated on FIG. 3A, with N_{c}=4096, Nₛ=2048 and δt=2 μs, there are almost 20 k-space samples per Nyquist voxel.

A Nyquist voxel is a voxel defined on the Cartesian grid in k-space, whose size Δk=1/FOV with FOV: Field of View in m (e.g. 20 cm=0.2m). Collecting a sample per Nyquist voxel allows for linear reconstruction in the central region of k-space without aliasing

As mentioned above, such an oversampling of the low-spatial frequencies is suboptimal as these extra k-space samples could instead be used to sample other higher frequencies. Moreover, as the k-space data are acquired as an integration over different k-space trajectories with different signal amplitude, repeated sampling of the same Nyquist voxels by different trajectories can lead to accumulation of B₀ artifacts. This can be seen on FIG. 3B, showing the mean and standard deviation of k-space data.

According to the present invention (“GoLF-SPARKLING”), the problem of the low-spatial frequency oversampling is solved by using modified SPARKLING-like trajectories which, in a central region of the k-space, define a Cartesian sampling.

In an exemplary embodiment of the invention, illustrated on FIG. 4, a sphere S of radius rₛ is defined in the center of the k-space. The entire k-space trajectory (panel A of the figure) is split into a non-Cartesian part, for instance a SPARKLING trajectory, outside the sphere (panel B) and a Cartesian part (panel C). In the displayed example N_{c}=256 and Nₓ=N_{y}=N_{z}=Ñ=64. The inner region of the k-space, inside the sphere S, is sampled by straight lines oriented in the kₓ direction, which are arranged according to a Cartesian grid in the k_{y}k_{z} plane. Panels D and E of FIG. 4 show cuts of the sphere S according to the k_{y}k_{z} and the kₓk_{z} plane, respectively. The pixel size in of the Cartesian grid in k-space is 2/Ñ according to each dimension.

As each k-space shot forms a Cartesian line, the area in the central slice with Cartesian sample circle is given by

$$Area = π {r}_{s}^{2} ≅ {( \frac{2}{\tilde{N}} )}^{2} {N}_{c} \qquad ( 8 )$$

from which one obtains

$${r}_{s} = \frac{2}{\tilde{N}} ⌊ \sqrt{\frac{{N}_{c}}{π}} ⌋ \qquad ( 9 )$$

Based on the Nyquist criteria, it is then necessary to sample at least every

$$Δ {k}_{x} = \frac{2}{\tilde{N}}$$

to have non-aliased reconstructed images. However, as the k-space data is sampled at every dwell time δt<Δt, in practice, the scanner can play k-space trajectory to take at least

$$\frac{Δ {k}_{x} Δ t}{δ t}$$

at every Δt. Generalizing this, it is possible to introduce k-space velocity at center of k-space as a dimensionless parameter v≥0, which is the number of Nyquist voxel steps Δkₓ taken in the readout direction kₓ per gradient raster time Δt. Particularly, k-space trajectory takes steps of size at

$$v Δ {k}_{x} < \frac{Δ {k}_{x} Δ t}{δ t}$$

at every Δt. The purpose of v is to efficiently utilize the gradient hardware in traversing the center of k-space at maximum feasible speed while maintaining Nyquist criteria after sampling at ADC at every δt. Taking a high value of v improves the k-space coverage, but can lower the signal-to-noise ratio as the MR signal is accumulated over a shorter time interval (see FIG. 6 and the corresponding discussion herebelow). Therefore, a tradeoff is needed. For instance, for T2* imaging at 3T, v≤1 is preferred.

If, without loss of generality, it is assumed that the Cartesian portion of the i^{th} k-space shot kᵢ starts as a k-space line at

$${x}_{i}^{s}$$

and ends at

$${x}_{i}^{e}$$

(see the panel E of FIG. 4), then the number of sample at the center of the k-space (i.e. inside the sphere S) of this shot is given by

$${N}_{i}^{k} = ⌈ \frac{{x}_{i}^{e} - {x}_{i}^{s}}{v Δ {k}_{x}} ⌉ \qquad ( 10 )$$

The k-space locations of these Nyquist points are given by

$${x}_{i}^{ℓ} = {x}_{i}^{s} + \frac{ℓ ( {x}_{i}^{e} - {x}_{i}^{s} )}{{N}_{i}^{K}} ∀ ℓ ∈ \{ 1 … {N}_{i}^{K} \} \qquad ( 11 )$$

This results

$${c}_{i} = {N}_{i}^{K}$$

constraints which are applied to the i^{th} space shot kᵢ at

$${N}_{i}^{K}$$

indices between

$${k}_{i}^{L} = ⌊ \frac{{N}_{c} - {N}_{i}^{K}}{2} ⌋ and {k}_{i}^{L} = ⌊ \frac{{N}_{c} + {N}_{i}^{K}}{2} ⌋$$

where “L” and “H” stand for “low” and “high” respectively. The constraints are then expressed by:

$${a}_{i , j}^{d} [ k ] = \{ \begin{matrix}1 for k = {k}_{i}^{L} + j ; j ∈ \{ 1 , … , {c}_{i} \} ; i ∈ \{ 1 , … , {N}_{c} \} ; d \{ x , y , z \} \\ 0 otherwise\end{matrix} \qquad ( 12 )$$

$${b}_{i , ℓ} = {x}_{i}^{ℓ} ∀ ℓ ∈ \{ 1 … {N}_{i}^{K} \} \qquad ( 13 )$$

The set of affine constraints of equations (12) and (13) allows performing Cartesian sampling of a central region of k-space S. In order to enforce that said Cartesian sampling is performed at the Nyquist rate, however, a suitable target sampling density (TSD) has to be chosen, which is different in the central region of the k-space and outside it.

Let us consider a normalized sampling domain Ω=[−1, 1]³. According to an exemplary embodiment, the target sampling density can be parameterized with cutoff C and decay D as follows:

$${Π}_{{r}_{s} , D} ( x ) = \{ \begin{matrix}{κ}_{1} & \vert x \vert < {r}_{s} \\ {{κ}_{2} ( \frac{{r}_{s}}{\vert x \vert} )}^{D} & \vert x \vert > {r}_{s}\end{matrix} \qquad ( 14 )$$

If N=N_{c}×Nₛ is the total number of sampling points, the number of samples in the central region S of k space defined by |x|<rₛ is

$${N}_{\vert x \vert < {r}_{s}} = N {κ}_{1} \frac{4}{3} π {r}_{s}^{3} \qquad ( 15 )$$

Additionally, the number of Nyquist points in the central region of the k-space is given by

$${N}_{N y q} = \frac{\frac{4}{3} {πr}_{s}^{3}}{Δ {k}_{x}^{3}} \qquad ( 16 )$$

If the trajectory velocity, defined above, is given by v, then there are

$$\frac{{N}_{N y q}}{v}$$

in the central region of the k-space. From the previous equations, one obtains:

$$\begin{matrix}{N}_{\vert x \vert < {r}_{s}} = \frac{{N}_{Nyq}}{v} \\ N {κ}_{1} \frac{4}{3} π {r}_{s}^{3} = \frac{\frac{4}{3} {πr}_{s}^{3}}{v Δ {k}_{x}^{3}} \\ {κ}_{1} = \frac{1}{vN Δ {k}_{x}^{3}}\end{matrix} \qquad ( 17 )$$

The ratio of number of Nyquist samples with non-Cartesian sampling (N^{nc}) and with Cartesian sampling (N^{c}) is given by the inverse of the ratio of the corresponding volumes:

$$\frac{{N}^{nc}}{{N}^{c}} = \frac{Δ {k}_{x}^{3}}{\frac{4}{3} {π ( \frac{Δ {k}_{x}}{2} )}^{2}} = \frac{6}{π} \qquad ( 18 )$$

From (18), and taking into account that the density needed for Cartesian sampling is νκ₁, one finds

$${κ}_{2} = v {κ}_{1} \frac{{N}^{n c}}{{N}^{c}} = v {κ}_{1} \frac{6}{π} \qquad ( 19 )$$

However, in practice one cannot achieve Poisson disk sampling due to constraints on speed and acceleration of the trajectory. To prevent any k-space hole, it is preferred to sample the non-Cartesian region at 1.5 times the Nyquist period, therefore taking

$${κ}_{2} = v v {κ}_{1} \frac{9}{π} \qquad ( 20 )$$

Finally, as Πᵣ_{ₛ}_{,D}(x) is a distribution, it has to be normalized, therefore its sum must be equal to 1:

$$\begin{matrix}{\int }_{0}^{1} {\int }_{0}^{2 π} {\int }_{0}^{π} {Π}_{{r}_{s} , D} ( x ) {x}^{2} sin θ d θ d ϕ dx = 1 \\ 4 π {\int }_{0}^{1} {Π}_{{r}_{s} , D} ( x ) {x}^{2} d x = 1 \\ 4 π ( {\int }_{0}^{{r}_{s}} {κ}_{1} {x}^{2} d x + {\int }_{{r}_{s}}^{1} {{κ}_{2} ( \frac{{r}_{s}}{\vert x \vert} )}^{D} {x}^{2} d x ) = 1 \\ 4 π ( \frac{{κ}_{1} {r}_{s}^{3}}{3} + {κ}_{2} \frac{{( {r}_{s} )}^{D} - {r}_{s}^{3}}{3 - D} ) = 1\end{matrix} \qquad ( 21 )$$

By replacing (19) and (20) into (21), one can solve iteratively for D.

A comparison between the left panel of FIG. 5 (MRI image of a test pattern—NIST phantom—obtained using prior art 3D SPARKLING trajectories) and its right panel (MRI image of the same pattern obtained using GoLF-SPARKLING trajectories) show that the invention allows obtaining a substantially better flat contrast.

GoLF-SPARKLING can advantageously be combined with the MORE-SPARKLING technique, described in European Patent Application 22305592.2 filed by the applicant on 21 Apr. 2022 and in [Chaithya et al. 2022]. This technique modifies the SPARKLING repulsion term by introducing in each individual term a weight W which increase with the temporal separation |tᵢ-tⱼ| of the sampling points along the trajectory in k-space:

$${F}_{p}^{r} ( K ) = \frac{1}{2 {p}^{2}} {\sum }_{1 \leq i , j \leq p} W ( \vert {t}_{i} - {t}_{j} \vert ) ‖ K [ i ] - K [ j ] ‖ \qquad ( 22 )$$

Preferably, W is an exponential function of |tᵢ-tⱼ|:

$$W ( \vert {t}_{i} - {t}_{j} \vert ) = exp ( \frac{\vert {t}_{i} - {t}_{j} \vert τ}{p} ) \qquad ( 23 )$$

where τ≥1 is a scalar parameter.

The weight W ensure that the resulting k-space trajectories are temporally homogeneous—i.e. that regions of the k-space that are close to each other are sampled at close times. The value of parameter τ has to be chosen (e.g. by grid-searching) to enforce temporally smooth k-space sampling, while avoiding undesirable k-space holes. Combining MORE-SPARKLING and GoLF-SPARKLING (MORE+GoLF SPARKLING) does not pose any specific difficulty because the first modify the cost function of the SPARKLING trajectory-design algorithm and the second the affine constraints onto which the trajectories are projected and the TSD.

FIG. 6 illustrates experimental results obtained for MORE+GoLF-SPARKLING trajectories with different values of the dimensionless k-space velocity: v=0.6 (panel i), 1 (ii), 2 (iii) and 3 (iv). The NIST phantom can help optimally tune v as it embodies resolution insets, which can be used to quantify image quality. For the sake of comparison, results obtained using a conventional “GRAPPA p4” k-space trajectory implementing Cartesian sampling (A), “pure” SPARKLING with t=0 (B), MORE SPARKLING with τ=1 (C), and GoLF SPARKLING with v=1 (D) are also presented. For the MORE-SPARKLING trajectory, some artifacts are observed near the regions involving change in the value of T*2 (marked with arrows in panel B). These artifacts are significantly reduced with GoLF (panel D). Further, in GoLF+MORE-SPARKLING (panels I-iv), it can be seen in the zoomed-in resolution insets that using v>1 causes significant losses in the details. This careful analysis indicates that v≤1 is preferable for T2*-w imaging at 3T. Within this range, for the sake of broader k-space coverage, the preferred choice is v=1, that is, the highest possible velocity to spend minimum time in the center of k-space and collect more samples in the high frequency region, resulting in sharper edges.

FIG. 7 shows in vivo results for “pure” SPARKLING with τ=0 (C), MORE SPARKLING with τ=1 (D), GoLF SPARKLING with v=1 (E) and MORE+GoLF SPARKLING with τ=1 and v=1 (F). In all cases, an acceleration factor AF=15 has been used, leading to a scan time of 3 min 21 s. Panel A corresponds to a conventional “GRAPPA p4” k-space trajectory implementing Cartesian sampling, and panel B to the ΔB₀ field map. As expected, MORE-SPARKLING appears more robust than classic SPARKLING to ΔB₀ inhomogeneities and present minimized artifacts. GoLF+MORE-SPARKLING provides a clear gain in image quality with significant reduction in noise levels and closer contrast to the Cartesian reference, paving the way toward improved clinical use of the SPARKLING trajectories. However, the GoLF+MORE-SPARKLING trajectory is slightly less robust than MORE-SPARKLING to ΔB₀ inhomogeneities and some residual artifacts can be seen in FIG. 7F.

FIG. 8 show in vivo results for MORE-SPARKLING (column A) and GoLF+MORE-SPARKLING (column B) trajectories at acceleration factors AF=10 (line 1), 15 (2), 20 (3) and 40 (5). The aim of this experiment was to assess how image quality evolves as a function of scan time. Overall, it can be observed that the GoLF feature provides less noisy and more detailed images as compared to the sole MORE-SPARKLING trajectories. Indeed the image quality is preserved up to AF=15 for GoLF+MORE-SPARKLING trajectories while degradation is already observed at AF=15 for MORE-SPARKLING alone. Additionally, a direct diagonal comparison can be drawn between the two approaches: image quality at AF=15, 20, 30, and 40 for GoLF+MORE-SPARKLING trajectories is comparable to that of MORE-SPARKLING trajectories at AF=10, 15, 20, and 30, respectively.

FIG. 9 illustrates, very schematically, an apparatus for carrying out a method according to the invention. This apparatus is basically a MRI scanner comprising a primary coil PC for generating the longitudinal field B₀, a single or preferably a plurality of radio-frequency coils TC for generating RF pulses and receiving the MRI signal (different radio-frequency coils may be used for transmission and reception, or the same coils may serve for both purposes), three gradient coils GCx, GCy and GCz for generating magnetic field gradients Gₓ, G_{y}, G_{z} along the x, y and z axes respectively (not represented). The gradient coils are disposed around a scanner bore SB in which a body BD to be imaged can be introduced. The term “body” should be construed broadly, designating any living or non-living object containing atomic nuclei at least some of which are supposed to have nonzero spin. For instance, body BD may be a human or animal body or a portion thereof (e.g. a head) or an article of manufacture such as a “phantom”. The apparatus also comprises a control unit CTR for driving said gradient coils in and said radio-frequency coil or coils according to a predetermined driving signals (reference DSR for the radio-frequency coils and DSG for the gradient coils) to implement a readout sequence (see e.g. FIG. 1) and a k-space trajectory of the GoLF-SPARKLING type. The apparatus also comprises a signal processing unit SPU for acquiring MRI signals from the radio-frequency coil or coils and performing image reconstruction. The control unit and the signal processing unit may be separate devices or a single device may implement both functions. Said device or devices may include one or more suitably programmed general purpose computers or digital signal processors, suitably configured specialized digital circuits or both. The signal processing unit also includes a receiving circuit for acquiring MRI signals, comprising a signal amplifier, a sampler and an analog to digital (ADC) converter.

The scanner of FIG. 9 differs from the prior art essentially in that the controlling unit CTR is configured or programmed for driving the gradient coils in such a way that they define a GoLF-SPARKLING k-space trajectory. For instance, a driving signal DRG defining a GoLF-SPARKLING trajectory may be stored in a memory of said controlling unit.

The invention has been described with reference to specific examples, but is not limited to them. For instance, the predetermined sampling density may be different from that expressed by equations (14)-(21).

The invention applies to 3D imaging, but also to 2D multislice k-space sampling and to 4D imaging where acceleration can be largely increased.

Trajectory optimization can be initialized starting from any classic k-space filling support (including Cartesian lines, spirals, radial, . . . ), not only radial spokes as in the exemplary embodiments.

The shape of the central region of k-space where sampling is Cartesian needs not be spherical or ellipsoidal, even if these constitute preferred embodiments. It can have other shapes, e.g. that of a cube or a rectangular cuboid.

The inventive method may be adapted to any types of MR readout scheme segmented or single-shot, from GRE (cf. the detailed examples above), Turbo FLASH (also called MPRAGE for brain applications) to Spin Echo (SE) or Turbo Spin Echo (TSE), TrueFisp (also called bSSFP), EPI.

It may also be adapted to any types of MR sequence weighting T1, T2, T2*, ρ (Spin Density), fMRI (functional MRI) or BOLD MRI, and preparation including none-exhaustively, ASL (Arterial Spin Labelling), DWI (Diffusion weighting imaging) with all its variants (DTI, DSI, IVIM, Kurtosis, NODDI), MTR (Magnetization Transfer Ratio) of any type including CEST, quantitative MRI including simultaneous multiparametric techniques such as Quantitative Susceptibility Mapping (QSM), MRF (MR Fingerprinting), MR Angiography both static or dynamic. This includes more exotic MRI applications such as MR thermometry or Electromagnetic Property Tomography (EPT). It may also be applied to heteronuclear imaging such as Sodium or Phosphorus.

It is compatible with parallel imaging using coil phased array and Simultaneous Multislice technique.

According to a different embodiment, Cartesian sampling can be performed in one or more regions of the k-space, other than a central region thereof.

### REFERENCES

[Andersson et al. 2003] J. L. R. Andersson, S. Skare, J. Ashburner “How to correct susceptibility distortions in spin-echo echo-planar images: application to diffusion tensor imaging”. Neurolmage, 20(2):870-888, 2003.
[Boyer et al, 2016] Boyer, Claire, et al. “On the generation of sampling schemes for magnetic resonance imaging.” SIAM Journal on Imaging Sciences 9.4 (2016): 2039-2072.
[Chaithya et al. 2020] Chaithya G R et al. “Optimizing full 3D SPARKLING trajectories for high-resolution T2*-weighted MagneticResonance Imaging. 2020 arXiv preprint arXiv:2108.02991.
[Chaithya et al. 2022] Chaithya G R et al. «MORE-SPARKLING: Non-Cartesian trajectories with Minimized Off-Resonance Effects”, Proceedings of the Joint Annual Meeting ISMRM-ESMRMB 2022 & ISMRT Annual Meeting, London, UK, 7-12 May 2022.
[Chauffert et al. 2016] Chauffert, Nicolas et al. “A projection algorithm for gradient waveforms design” in Magnetic Resonance Imaging. IEEE Transactions on Medical Imaging, Institute of Electrical and Electronics Engineers, 2016, 35 (9), pp. 2026-2039.
[Daval-Frerot et al. 2021] Daval-Frérot, Guillaume “Off-resonance correction of non-Cartesian SWI using internal field map estimation” International Society for Magnetic Resonance in Medicine, May 2021, Online, United States.
[Haldar, 2014] Haldar, Justin P. “Low-Rank Modeling of Local k-Space Neighborhoods (LORAKS) for Constrained MRI”, IEEE Transaction of Medical Imaging 33.3 (2014): 668-680.
[Lazarus et al, 2017] Lazarus, Carole, et al. “SPARKLING: Novel Non-Cartesian Sampling Schemes for Accelerated 2D Anatomical Imaging at 7T Using Compressed Sensing.” 25th annual meeting of the International Society for Magnetic Resonance Imaging, April 2017, Honolulu, United States.
[Lazarus 2019] Lazarus, Carole et al. “SPARKLING: variable-density k-space filling curves for accelerated T 2*-weighted MRI” Magnetic Resonance in Medicine, Wiley, 2019, 81 (6), pp. 3643-3661.
[Lustig et al, 2007] Lustig M., Donoho D., Pauly J. M. Sparse MRI: The application of compressed sensing for rapid MR imaging. Magn Reson Med 2007; 58:1182-1195
[Sutton et al. 2003] Sutton, Bradley P, et al. “Iterative Image Reconstruction for MRI in the Presence of Field Inhomogeneities”. IEEE Transactions on Medical Imaging 2003; 22 (2): 178-88.

1. A method of performing magnetic resonance imaging of a body using a magnetic resonance imaging scanner comprising a scanner bore (SB), a primary coil (PC), radio frequency coils (RFC), gradient coils (GCₓ, GC_{y}, GC_{z}) and a signal-processing unit (SPU), the method comprising the steps of:
a. positioning the body (BD) in a scanner bore (SB) where a static and substantially uniform magnetic field (B₀), called longitudinal field, oriented along a direction (z), called longitudinal direction, is established by the primary coil (PC);
b. using all or part of the radio-frequency coils (RFC) to transmit to said body at least one radio-frequency pulse (RFP) adapted to excite nuclear spins inside said body;
c. after said or each said radio-frequency pulse, using the gradient coils (GCₓ, GC_{y}, GC_{z}) to apply to said body time-varying magnetic field gradients (Gₓ, G_{y}, G_{z}) defining a trajectory (ST) in k-space and simultaneously using all or part of the radio-frequency coils to acquire samples of a magnetic resonance signal emitted by the excited nuclear spin, each sample corresponding to a point (KS) of the k-space belonging to said trajectory; and
d. using the signal processing unit (SPU) to apply a nonlinear reconstruction algorithm to the acquired samples to reconstruct a magnetic resonance image of said body;
wherein said trajectory (ST, MT) in k-space is a continuous trajectory complying with a set of constraints including constraints on maximum amplitude and maximum slew rate of said time-varying magnetic field gradients, such that the points (KS) of the k-space corresponding to the samples, called sampling points, define a pseudo-random sampling of the k-space, approximating a predetermined target sampling density, said trajectory in k-space minimizing, subject to said set of constraints, a cost function defined by the difference between a first term, called attraction term, promoting adherence of the distribution of sampling points in k-space to said predetermined target sampling density, and a second term, called repulsion term, promoting separation in k-space between sampling points, said repulsion term being expressed as a sum of contributions corresponding to respective pairs of sampling points;
wherein said trajectory (ST, MT) in k-space defines a Cartesian sampling of a central region(S) of the k-space.

2. The method of claim 1, wherein said set of constraints comprise a affine constraint imposing said Cartesian sampling of said central region(S) of the k-space.

3. The method of claim 1, wherein said central regions(S) of the k-space has a spherical or ellipsoidal shape.

4. The method of claim 1, wherein the predetermined target sampling density is such that said trajectory is suitable for sampling said central region of the k-space at a rate comprised between the Nyquist rate and 1.5 times the Nyquist rate.

5. The method of claim 1, wherein said attraction term is defined by
$${F}_{p}^{a} ( K ) = {argmin}_{K ∈ {Q}^{Nc}} \frac{1}{p} {\sum }_{i = 1}^{p} {\int }_{Ω} ‖ x - K [ i ] ϱ ( x ) ‖ dx$$
and said repulsion term is defined by
$${F}_{p}^{r} ( K ) = \frac{1}{2 {p}^{2}} {\sum }_{1 \leq i , j \leq p} w ( \vert {t}_{i} - {t}_{j} \vert ) ‖ K [ i ] - K [ j ] ‖$$
where:
K is a two or three dimensional vector representing the coordinates of a sampling point in k-space;
N=N_{c}·Nₛ is the number of sampling points along the trajectory, each trajectory being constituted of N_{c}≥1 shots each comprising Nₛ>1 sampling points;
Q^{N}^{_{c}} is a set of curves in k-space comprising N_{c} segments of Nₛ points each and complying with said constraints;
Ω is a sampling region in k-space;
X represents a generic point of the sampling region Ω;
ρ is said predetermined target sampling density;
∥⋅∥ is a norm;
tᵢ, tⱼ are the times at which two sampling points of a same shot, identified by integer indices i and j, are reached; and
W is a monotonously increasing function of |tᵢ-tⱼ| expressing said weight.

6. The method of claim 1, further comprising a preliminary step of computing said trajectory (ST) in k-space.

7. A computer-implemented method of computing a trajectory in k-space to be used for sample acquisition in magnetic resonance imaging, the method comprising the steps of:
i. determining, or receiving from a user input, a number N_{c}≤1 l of shots constituting the trajectory and a number Nₛ>1 of sampling points along each said shot;
ii. determining, or receiving from a user input, a predetermined target sampling density;
iii. determining, or receiving from a user input, a set of constraints including constraints on amplitudes of discrete time derivatives of the trajectory;
iv. computing said trajectory by minimizing, subject to said set of constraints, a cost function defined by the difference between a first term, called attraction term, promoting adherence of the distribution of sampling points in k-space to said predetermined target sampling density, and a second term, called repulsion term, promoting separation in k-space between sampling points, said repulsion term being expressed as a sum of contributions corresponding to respective pairs of sampling points;
wherein said trajectory (ST, MT) in k-space defines a Cartesian sampling of a central region(S) of the k-space.

8. The method of claim 7, wherein said set of constraints comprise a affine constraint imposing said Cartesian sampling of said central region(S) of the k-space.

9. The method of claim 8, wherein said central regions(S) of the k-space has a spherical or ellipsoidal shape.

10. The method of claim 7, wherein the predetermined target sampling density is such that said trajectory is suitable for sampling said central region of the k-space at a rate comprised between the Nyquist rate and 1.5 times the Nyquist rate.

11. The method of claim 7, wherein said attraction term is defined by
$${F}_{p}^{a} ( K ) = {argmin}_{K ∈ {Q}^{Nc}} \frac{1}{p} {\sum }_{i = 1}^{p} {\int }_{Ω} ‖ x - K [ i ] ϱ ( x ) ‖ dx$$
and said repulsion term is defined by
$${F}_{p}^{r} ( K ) = \frac{1}{2 {p}^{2}} {\sum }_{1 \leq i , j \leq p} w ( \vert {t}_{i} - {t}_{j} \vert ) ‖ K [ i ] - K [ j ] ‖$$
where:
K is a two or three dimensional vector representing the coordinates of a sampling point in k-space;
N=N_{c}·Nₛ is the number of sampling points along the trajectory;
Q^{N}^{_{c}} is a set of curves in k-space comprising N_{c} segments of Nₛ points each and complying with said constraints;
Ω is a normalized sampling region in k-space;
x represents a generic point of the sampling region Ω;
ρ is said predetermined target sampling density;
∥⋅∥ is a norm;
tᵢ, tⱼ are the times at which two sampling points of a same shot, identified by integer indices i and j, are reached; and
W is a monotonously increasing function of |tᵢ-tⱼ| expressing said weight.

12. A computer program comprising instructions which, when the program is executed by a computer, cause the computer to carry out the method of claim 7.

13. A set of driving signals (DSG) for gradient coils of a magnetic resonance imaging scanner which, when applied to said gradient coils, drive them to generate time-varying magnetic field gradients (Gₓ, G_{y}, G_{z}) defining a trajectory (ST, MT) in k-space passing through a plurality of points (KS) called sampling points defining a pseudo-random sampling of the k-space, approximating a predetermined target sampling density; wherein said trajectory:
is continuous and complies with a set of constraints including constraints on maximum amplitude and maximum slew rate of said time-varying magnetic field gradients;
minimizes, subject to said set of constraints, a cost function defined by the difference between a first term, called attraction term, promoting adherence of the distribution of sampling points in k-space to said predetermined target sampling density, and a second term, called repulsion term, promoting separation in k-space between sampling points, said repulsion term being expressed as a sum of contributions corresponding to respective pairs of sampling points;
wherein said trajectory (ST, MT) in k-space defines a Cartesian sampling of a central region(S) of the k-space.

14. A magnetic resonance imaging scanner comprising:
a scanner bore (SB) inside which a body can be positioned;
a primary coil (PC) configured to establish in the scanner bore a static and substantially uniform magnetic field (B₀), called longitudinal field, oriented along a direction (z), called longitudinal direction;
radio frequency coils (RFC) configured to transmit to said body at least one radio-frequency pulse (RFP) adapted for exciting nuclear spins inside said body and to acquire samples of a magnetic resonance signal emitted by the excited nuclear spin;
gradient coils (GCₓ, GC_{y}, GC_{z}) configured to apply to said body time-varying magnetic field gradients (Gₓ, G_{y}, G_{z}) defining a trajectory (ST, MT) in k-space;
a signal processing unit (SPU) to apply a nonlinear reconstruction algorithm to the acquired samples of the magnetic resonance signal for reconstructing a magnetic resonance image of said body; and
a controller (CTR) configured to generate driving signals (DSR) for the radio frequency coils and (DSG) for the gradient coils;
wherein the controller is configured to generate said driving signals for (DSG) for the gradient coils to generate a said time-varying magnetic field gradients (Gₓ, G_{y}, G_{z}) such that said a trajectory (ST) in k-space passes through a plurality of points (KS) called sampling points defining a pseudo-random sampling of the k-space, approximating a predetermined target sampling density, said trajectory being continuous and compliant with a set of constraints including constraints on maximum amplitude and maximum slew rate of said time-varying magnetic field gradients, said trajectory in k-space also minimizing, subject to said set of constraints, a cost function defined by the difference between a first term, called attraction term, promoting adherence of the distribution of sampling points in k-space to said predetermined target sampling density, and a second term, called repulsion term, promoting separation in k-space between sampling points, said repulsion term being expressed as a sum of contributions corresponding to respective pairs of sampling points;
and wherein the controller is also configured to generate driving signals (DSR) for the radio frequency coils such that they acquire said samples of a magnetic resonance signal emitted by the excited nuclear spin in correspondence to the sampling points (KS) of the trajectory in k-space;
wherein said trajectory (ST, MT) in k-space defines a Cartesian sampling of a central region (S) of the k-space.
